(19) United States
(12) Patent Application Publication
(10) Pub. No.: US 2007/0118006 A1
(43) Pub. Date: May 24, 2007

(54) METHOD OF MAKING AROMATIC PRODUCTS

(76) Inventor: Garo Garbis VAPORCIYAN, Houston, TX (US)

Correspondence Address: SHELL OIL COMPANY, P O BOX 2463, HOUSTON, TX, 772522463, US

(21) Appl. No.: 11/532,020

(22) Filed: Sep. 14, 2006

Related U.S. Application Data
(60) Provisional application No. 60/717,706, filed on Sep. 16, 2005.
(60) Provisional application No. 60/717,988, filed on Sep. 16, 2005.

(51) Int. Cl. C07C 15/12; C07C 5/22; C07C 5/52

(52) U.S. Cl. 585/475

(57) ABSTRACT

A method of making an alkylaromatic compound which comprises contacting an alkylatable aromatic hydrocarbon and a polyalkylaromatic compound in a transalkylation system to yield an alkylaromatic compound wherein at least a portion of the polyalkylaromatic compound has been formed by contacting an alkylatable aromatic hydrocarbon and an olefin at a location that is remote from the transalkylation system. The method may further comprise further reacting the alkylaromatic compound to produce one or more aromatic products.

This application claims the benefit of U.S. Provisional Application No. 60/717,706, filed Sep. 16, 2005 and U.S. Provisional Application No. 60/717,988, filed Sep. 16, 2005, which are hereby incorporated by reference.

The invention relates to a method of making an alkylaromatic compound from an alkylatable aromatic hydrocarbon and a polyalkylaromatic compound. In certain embodiments the invention further relates to a method of making an aromatic product by further reacting an alkylaromatic compound.

Aromatic compounds may be alkylated to form alkylaromatic compounds that are capable of being further reacted to form aromatic products, for example alkenylaromatic compounds, phenolic compounds and/or other products. The product of an alkylation system may also include polyalkylaromatic compounds that may be subjected to transalkylation to yield monoalkylated aromatic compounds that may then be further reacted to form aromatic products.

Disclosed in U.S. Pat. No. 3,525,776 is a process for producing an alkenylaromatic hydrocarbon by alkylating an alkylatable aromatic hydrocarbon followed by dehydrogenation of the resulting monoalkylated aromatic compound. The disclosed process includes the use of an alkylation reactor, a transalkylation reactor, and a dehydrogenation reactor. The process provides for the separation of the polyalkylated aromatic compounds and their recycle to the transalkylation reactor to convert them to monoalkylated aromatics that may suitably be subjected to a dehydrogenation step to yield an alkenylaromatic compound.

U.S. Pat. No. 4,169,111 discloses the alkylation of benzene with ethylene in an alkylation zone. A portion of the diethylbenzene produced in the alkylation zone is recycled to that alkylation zone to improve catalyst life. The remainder of the diethylbenzene and the other polyethylbenzenes are fed to a transalkylation reactor to be converted to ethylbenzene.

It is an object of the invention to improve the economics of manufacturing aromatic products. Another object of the invention is to reduce the amount of alkylatable aromatic hydrocarbon that is shipped from one location to another in the manufacturing chain of aromatic products.

Yet, another object of the invention is to provide a means by which low cost olefins produced in certain geographic locations that are remote from an alkylaromatic reaction system, may be economically used as an indirect input to said alkylaromatic reaction system. In the present context, an alkylaromatic reaction system is a system that provides for the reaction of alkylaromatic compounds to produce one or more aromatic products.

Thus, in accordance with the invention, a method is provided which comprises contacting an alkylatable aromatic hydrocarbon and a polyalkylaromatic compound in a transalkylation system to yield an alkylaromatic compound wherein at least a portion of the polyalkylaromatic compound has been formed by contacting an alkylatable aromatic hydrocarbon and an olefin at a location that is remote from the transalkylation system.

In accordance with another embodiment of the invention, a method is provided which comprises reacting an alkylaromatic compound to yield an alkenylaromatic compound in a reaction system wherein at least a portion of the alkylaromatic compound has been formed in a transalkylation system by contacting an alkylatable aromatic hydrocarbon and a polyalkylaromatic compound that has been formed at a location that is remote from the transalkylation system.

In accordance with yet another embodiment of the invention, a method is provided which comprises reacting an alkylaromatic compound to yield a phenolic compound in a reaction system wherein at least a portion of the alkylaromatic compound has been formed in a transalkylation system by contacting an alkylatable aromatic hydrocarbon and a polyalkylaromatic compound that has been formed at a location that is remote from the transalkylation system.

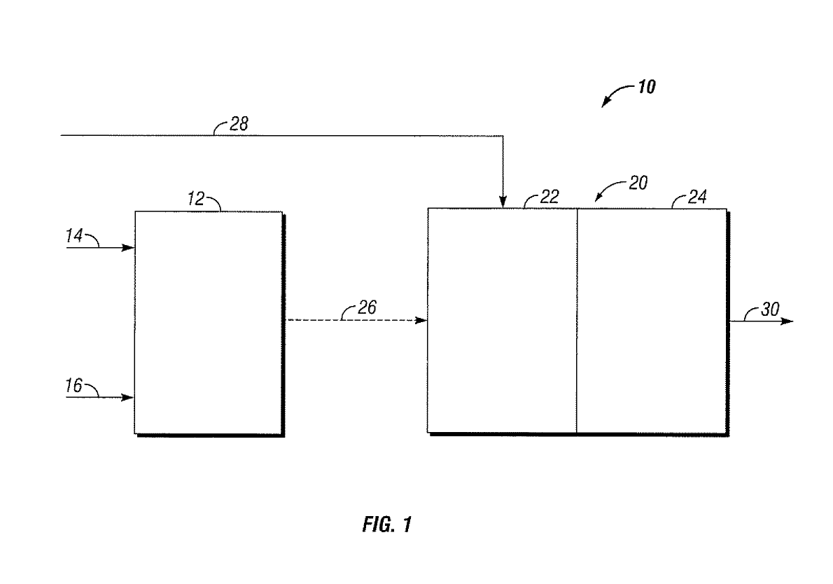

FIG. 1 is a simplified schematic diagram of an embodiment of the inventive process for making aromatic products.

The inventive method provides for the economically advantageous location of an alkylaromatic reaction system remotely from the sources of the olefin feedstock inputs required for the manufacture of its alkylaromatic feed. There are numerous advantages that are derivable from remotely locating an alkylation system from the location of the alkylaromatic reaction system and/or remotely from a transalkylation system. One such advantage is that the location of separate elements of the manufacturing chain at locations based on the availability of the various feedstocks may provide for the use of cost advantaged feedstocks Thus, for example, an alkylation system may be located near to the sources of cost advantaged olefins where such cost advantaged olefins are used as a feed input to an alkylation system, while the alkylaromatic reaction system is located near to the ultimate end-user of the product of the alkylaromatic reaction system that may be at a location remote from the sources of the olefin.

The inventive method may include the process steps of alkylation of an alkylatable aromatic hydrocarbon to form a polyalkylaromatic compound followed by the transalkylation of the polyalkylaromatic compound to form an alkylaromatic compound that may be followed by a further reaction of the alkylaromatic compound to yield one or more aromatic products. The alkylation may include passing a feed, which includes an alkylatable aromatic hydrocarbon and an olefin, to an alkylation reactor that provides for the production of a polyalkylaromatic compound.

The alkylatable aromatic hydrocarbon of the feed to the alkylation reactor may include any suitable alkylatable aromatic hydrocarbon including various substituted benzene compounds as well as benzene. Examples of such alkylatable aromatic hydrocarbons include benzene, toluene, ethylbenzene, the propylbenzenes, the butylbenzenes, the xylenes, the diethylbenzenes, the dipropylbenzenes, the dibutylbenzenes, and possibly higher molecular weight alkylaromatic hydrocarbons. It is preferred for the alkylatable aromatic compound used as a feed to the alkylation reactor to be selected from the group of aromatic compounds consisting of benzene, toluene, ethylbenzene, and propylbenzene. Benzene is the most preferred alkylatable aromatic hydrocarbon.

The olefin to be fed along with the alkylatable aromatic hydrocarbon to the alkylation reactor may include any monoolefin that is capable of reacting with the alkylatable aromatic hydrocarbon of the alkylation reactor feed including monoolefins having from two to five carbon atoms. Specific examples of possible olefins include ethylene, propylene, the butenes, the pentenes and mixtures of any combination thereof, such as, for example, mixtures of ethylene and propylene Thus, the alkylation reactor feed may comprise an olefin, which may comprise ethylene, or propylene, or ethylene and propylene. The olefin may comprise internal olefins and/or alpha olefins. The internal olefins may comprise 2-butene and/or 2-pentene. The alpha olefins may comprise ethylene, propylene, and/or 1-butene.

As noted above, the alkylation system may include an alkylation reactor for receiving an alkylation feed; and, it yields a polyalkylaromatic compound. This alkylation reactor may be any suitable apparatus that provides for the contacting of the alkylation feed with an alkylation catalyst under reaction conditions that are suitable for the alkylation of alkylatable aromatic hydrocarbons of the alkylation system feed. The apparatus may include a catalytic distillation apparatus Thus, generally, the alkylation reactor provides means for contacting the alkylation feed under suitable alkylation reaction conditions and may include a vessel that defines a reaction zone containing the alkylation catalyst.

An aspect of the inventive method is for the alkylatable aromatic hydrocarbon to be alkylated to form a polyalkylaromatic compound. As used herein, the term polyalkylaromatic compound refers to a substituted benzene molecule wherein at least two of the six carbon atoms of the benzene ring have bonded alkyl groups. Thus, the polyalkylaromatic compound may include a benzene ring substituted with two alkyl groups or three alkyl groups. The use of an alkylation step that preferentially yields polyalkylaromatic compounds as opposed to yielding monoalkylaromatic compounds, may provide significant economic, safety and other advantages. One such advantage is that it allows for the economically advantageous placement of the alkylation system at a location that is near sources of olefin feedstocks even though such sources may be geographically remote from sources of alkylatable aromatic hydrocarbons and the locations of alkylaromatic reaction systems The inventive method provides for these advantages by reducing the amount of alkylatable aromatic hydrocarbon required to be transported to the site of the alkylation system for use as a feed; since, the alkylatable aromatic hydrocarbon used is loaded-up with olefin by the alkylation reaction.

The higher the level of alkylation of the alkylatable aromatic hydrocarbon feed, the less alkylatable aromatic hydrocarbon feed that is required to be delivered to the alkylation site and the more olefin that can be transported from the alkylation site to the transalkylation site and the alkylaromatic reaction site by way of transportation of the polyalkylaromatic compound produced by the alkylation system To illustrate this, reference is now made to the schematic of FIG. 1, which depicts an aromatic product manufacturing chain 10 according to an embodiment of the inventive method. Included in the manufacturing chain 10 is the alkylation system 12, which provides for the alkylation of an alkylatable aromatic hydrocarbon by an olefin. The olefin is fed to the alkylation system 12 by way of line 14, and the alkylatable aromatic hydrocarbon is fed to the alkylation system 12 by way of line 16. A polyalkylaromatic compound is yielded from the alkylation system 12 and is transported to an integrated system 20, which includes a transalkylation system 22 and an alkylaromatic reaction system 24, by way of transportation means 26. An alkylatable aromatic hydrocarbon is fed to the transalkylation system 22 by way of line 28. An aromatic product stream is yielded from the alkylaromatic reaction system 24 through line 30. The product stream may comprise one or more aromatic products.

It is understood that, in a material balance based on the stoichiometric yields (ignoring yield losses) across the overall manufacturing chain 10, for each mole of aromatic product yielded one mole of olefin and one mole of alkylatable aromatic hydrocarbon are required. If the alkylatable aromatic hydrocarbon feed to the alkylation system 12 is alkylated to form a polyalkylaromatic compound, such as a dialkylaromatic compound, instead of a monoalkylaromatic compound, then fewer moles of alkylatable aromatic hydrocarbon will be fed to the alkylation system 12 to be alkylated and fewer moles of monoalkylaromatic compound will be yielded from alkylation system 12 for each mole of aromatic product that is produced by the integrated system 20. However, the reduction in the amount of alkylatable aromatic hydrocarbon that is to be fed to the alkylation system 12 must be offset by an equal molar amount of alkylatable aromatic hydrocarbon that is fed to the transalkylation system 22 of the integrated system 20.This illustrates one of the advantages of the inventive method in that the alkylation step is utilized so as to reduce the amount of alkylatable aromatic hydrocarbon required for use in the alkylation step while increasing the amount of olefin per mole of alkylatable aromatic hydrocarbon reacted in the alkylation step, thus, providing possible economic benefits by improving the potential of locating an alkylation system near to a source of olefin but distant from a source of alkylatable aromatic hydrocarbon. In such a situation, the required volume of alkylatable aromatic hydrocarbon to be delivered to the alkylation system and the volume of polyalkylaromatic compound transported from the alkylation system are reduced by the alkylation reaction as compared to when the alkylation step is essentially only a monoalkylation reaction.

To further illustrate the beneficial features of the inventive method, reference is again made to FIG. 1. In the case in which only a monoalkylaromatic compound is yielded from the alkylation system 12, stoichiometrically, for each mole of monoalkylaromatic compound yielded from alkylation system 12, one mole of olefin and one mole of alkylatable aromatic hydrocarbon are reacted, and for each mole of the monoalkylaromatic compound that is charged to the integrated system 20 one mole of aromatic product is yielded. This is typically a desired result; however, with the inventive method, it is more desirable to yield from the alkylation system 12 a polyalkylaromatic compound as opposed to a monoalkylaromatic compound; and, with such yield of a polyalkylaromatic compound from the alkylation system 12, less alkylatable aromatic hydrocarbon is consumed in the alkylation system 12 to provide for a given overall yield of aromatic product by the manufacturing chain 10. The remaining alkylatable aromatic hydrocarbon required for the yield of the aromatic product is then added by its introduction to the transalkylation system 22 of the integrated system 20.

In one example of a shift in benzene usage, in the event that the alkylation system 12 yields dialkylbenzene, a molar balance across the manufacturing chain 10 indicates that for each mole of aromatic product yielded one-half (½) mole of benzene is fed to alkylation system 12 along with one mole of olefin to yield one-half (½) mole of dialkylbenzene. This one-half mole of dialkylbenzene is fed along with one-half (½) mole of benzene to the transalkylation system 22 whereby the transalkylation reaction therein provides for the yielding of one mole of monoalkylbenzene that is fed to the alkylaromatic reaction system 24 that yields one mole of aromatic product.

Another example of the shift in benzene usage is when the alkylation system 12 yields trialkylbenzene. In this case, the molar balance across the manufacturing chain 10 indicates that for each mole of aromatic product yielded one-third (⅓) mole of benzene is fed to the alkylation system 12 along with one mole of olefin to yield one-third (⅓) mole of trialkylbenzene. This one-third mole of trialkylbenzene is fed along with two-thirds (⅔) mole of benzene to the transalkylation system 22 whereby the transalkylation reaction provides for the yielding of one mole of monoalkylbenzene that is fed to the alkylaromatic reaction system 24 that yields one mole of aromatic product.

As may be discerned from the discussion above, for a given amount of aromatic product produced across the manufacturing chain 10, the inventive method allows for the shifting of the alkylatable aromatic hydrocarbon usage from one location to another. This is achieved by operation of an alkylation system in an operating mode that favors the production of polyalkylaromatic compounds as opposed to monoalkylaromatic compounds. This loading-up of the alkylatable aromatic hydrocarbon molecule with olefin provides for the conversion of the olefin into a form that is more easily transportable and minimizes the amount of alkylatable aromatic hydrocarbon that is required for the alkylation reaction as compared to the situation when only monoalkylaromatic compounds are produced. Thus, an advantage of the inventive method is that the alkylation system 12 may be placed in a geographic location that is close to sources of olefins, particularly near sources of low cost olefins, but which are distant from economical sources of alkylatable aromatic hydrocarbon or alkylaromatic reaction systems, or both. The transalkylation system 22 may then be placed at such geographic locations that are closer to economical sources of alkylatable aromatic hydrocarbon or sources of aromatic product demand that are remote from the aforementioned sources of olefins.

Any suitable alkylation catalyst known in the art may be used in the alkylation step, but it should be recognized that much of the art teaches the need in conventional alkylation systems for the alkylation catalysts to be selective toward the yield of singularly alkylated aromatic hydrocarbon, or monoalkylation, with a minimization of the amount of polyalkylation that occurs. However, for the instant method, contrary to the teachings expressed in much of the prior art, it is desirable for the alkylation catalyst to preferentially provide for the polyalkylation of an alkylatable aromatic hydrocarbon to yield a polyalkylaromatic compound. Suitable alkylation catalysts are disclosed in such patents as U.S. Pat. No. 3,525,776; U.S. Pat. No. 3,751,504; U.S. Pat. No. 3,763,259; U.S. Pat. No. 4,169,111; U.S. Pat. No. 4,393,262; U.S. Pat. No. 4,876,408; U.S. Pat. No. 5,081,323; U.S. Pat. No. 5,177,280; U.S. Pat. No. 5,243,116; U.S. Pat. No. 5,530,170; and U.S. Pat. No. 6,670,517, all of which are incorporated herein by reference.

Alkylation catalysts preferred for use as the alkylation catalyst of the inventive method are those that are selective toward the yield of polyalkylaromatic compounds, with a minimum yield of undesirable side products, such as, for example, ethylene, propylene, or butene oligomers or polymers. Undesirable side reactions include those where part of an alkyl side chain breaks off and those where ring closure leading to cyclics such as indanes and indenes may occur.

Generally, the alkylation catalyst may include a hydrogen halide, a boron halide, a metal halide, for example the halides of aluminum, zinc, iron or copper, and a crystalline aluminosilicate, all as described in detail in the aforementioned patents. Among the metal halide compounds, Friedel-Crafts type catalytic compounds, such as, aluminum chloride are preferred. Some of the more desirable alkylation catalysts for use in the inventive method comprise a crystalline aluminosilicate as at least one of its components. Various of the suitable types of crystalline aluminosilicates for use in the alkylation catalyst of the invention are described in detail in the aforementioned patents, and they may include intermediate pore size zeolites having a Constraint Index in the range of from about 1 to about 12 such as those that are designated as ZSM-5, ZSM-11, ZSM-12, ZSM-22, ZSM-23, ZSM-35, ZSM-48, ZSM-50, ZSM-57. The silica-to-alumina molar ratio of the zeolite used in the alkylation catalyst may exceed 12:1, and, preferably, it exceeds 30:1.

The alkylation catalyst may include a silica-bound ZSM-5 zeolite catalyst such as is described in U.S. Pat. No. 6,670,517, or an intermediate pore size zeolite that has been selectivated with a siliceous material as described in U.S. Pat. No. 5,530,170, or an acid-modified zeolite as described in U.S. Pat. No. 5,243,116, or a dealuminated Y zeolite as described in U.S. Pat. No. 5,177,280, or a zeolite beta catalyst as described in U.S. Pat. No. 5,081,323.

An aspect of the invention is for the alkylation reaction conditions under which the alkylation system feed, which comprises an alkylatable aromatic hydrocarbon and an olefin, is contacted with the alkylation catalyst to be such as to preferentially provide for the polyalkylation, as opposed to the monoalkylation, of the alkylatable aromatic hydrocarbon In the case of the polyalkylation of an alkylatable aromatic hydrocarbon by olefin, alkylation reaction conditions include a relatively low alkylatable aromatic hydrocarbon-to-olefin molar ratio (the alkylatable aromatic hydrocarbon-to-olefin molar ratio is hereinafter referred to as the “alkylation ratio”) in the alkylation system feed, since a low alkylation ratio promotes the polyalkylation of the alkylatable aromatic hydrocarbon. In the prior art alkylation processes, however, it is usually desirable to have a high alkylation ratio in the alkylation system feed due to the polyalkylation inhibiting effect of a high alkylation ratio. An example of such an unsuitably high alkylation ratio is one that exceeds 8.5:1, or an alkylation ratio that exceeds 3:1.

The low alkylation ratio of the feed to the alkylation step may provide further advantages over the conventional processes by lowering the costs associated with separation and recycle of the excess alkylatable aromatic hydrocarbon. There is also the potential for lower equipment costs of the alkylation system due to the smaller amounts of alkylatable aromatic hydrocarbon that are handled and recycled.

While it is recognized that the selectivity of the alkylation catalyst toward the yield of polyalkylaromatic compound and its other catalytic properties may influence the required alkylation ratio of the feed to the alkylation system, normally the ratio should be such as to promote the polyalkylation of alkylatable aromatic hydrocarbon, and, typically it is less than about 2.8:1. For better operation of the alkylation system, however, the alkylation ratio should be less than 2.5:1 and, preferably, less than 2:1. Most preferably, for the best yield of polyalkylaromatic compound, the alkylation ratio is less than 1.5:1.

Generally, the number of moles of olefin that are reacted per mole of alkylatable aromatic hydrocarbon in the polyalkylation reaction may be at least 1.5, preferably, the number of moles of olefin reacted per mole of alkylatable aromatic hydrocarbon reacted may be at least 1.8, and, most preferably, at least or about 2.

The lower limit of the alkylation ratio is determined by the practical process considerations taking into account such things as the performance properties of the alkylation catalyst and the separation capabilities of the process system and costs associated with the component separations. An excessively low alkylation ratio may result in promoting the undesirable polymerization of the olefin. Thus, the alkylation ratio in the alkylation system feed may exceed 0.1:1 and, preferably, exceed 0.2:1. It is most preferred for the alkylation ratio in the alkylation system feed to exceed 0.5:1. In view of the above, the alkylation ratio in the alkylation system feed may be in the range of from about 0.1:1 to about 2.8:1, more specifically, from 0.2:1 to 2.5:1. Preferably, the ratio is in the range of from 0.5:1 to 2:1.

The alkylation reaction may take place under liquid phase conditions or vapor phase conditions or mixed liquid and vapor phase conditions; provided, of course, that the alkylation reaction conditions are such that polyalkylation of the alkylatable aromatic hydrocarbon is induced. It is desirable to minimize the amount of monoalkylation reactions that occur while promoting the amount of polyalkylation reactions that occur. In directional terms, higher reaction temperature conditions will favor polyalkylation and lower reaction temperature conditions will favor monoalkylation.

Typical vapor phase alkylation reaction conditions for the polyalkylation of alkylatable aromatic hydrocarbon with olefin may include an alkylation reaction temperature in the range of from about 340° C. (644° F.) to 480° C. (896° F.), an alkylation reaction pressure in the range of from about atmospheric pressure to about 20,000 kPa (2900 psig), and a weight hourly space velocity (WHSV) based on the olefin of the alkylation system feed in the range of from about 0.5 hr⁻¹ to about 10 hr⁻¹. The mole ratio of the alkylatable aromatic hydrocarbon-to-olefin is that as described above. It is understood that the vapor phase alkylation reaction conditions for the polyalkylation of an alkylatable aromatic hydrocarbon with other olefins may be different from those disclosed above.

Typical liquid phase alkylation reaction conditions for the polyalkylation of an alkylatable aromatic hydrocarbon with an olefin may include an alkylation reaction temperature in the range of from about 25° C. (77° F.) to 450° C. (842° F.), an alkylation reaction pressure in the range of from about atmospheric pressure to about 20,600 kPa (3000 psig), and a weight hourly space velocity (WHSV) based on the olefin of the alkylation system feed in the range of from about 0.5 hr⁻¹ to about 10 hr⁻¹. The mole ratio of the alkylatable aromatic hydrocarbon-to-olefin is that as described above. It is understood that the liquid phase alkylation reaction conditions for the polyalkylation of an alkylatable aromatic hydrocarbon with other olefins may be different from those disclosed above.

It is a feature of the inventive method for the alkylation system to be physically remote from the transalkylation system. And, in fact, it is this aspect of the invention that allows for the economically advantageous physical location of the alkylation system near to the sources of low priced olefin supplies even when at such locations the supplies of alkylatable aromatic hydrocarbons may be limited. Thus, statements herein concerning the location of the alkylation system as being remotely located from other process systems mean that typically the alkylation system is at least not in fluid communication with either the transalkylation system or the alkylaromatic reaction system. A lack of fluid communication, thus, does not permit the direct transfer of the polyalkylaromatic compound to either the transalkylation system or the alkylaromatic reaction system, or to both. Instead, due to the remote location of the alkylation system, its polyalkylaromatic compound must be transferred by any suitable transportation means to the location of the transalkylation system that uses the polyalkylaromatic compound as a transalkylation feed.

It is considered herein that the references to remote location may mean that there is a distance separating the alkylation system and the transalkylation system of at least 80 km (50 miles), but the distance may be greater than 300 km (186 miles) or even greater than 800 km (497 miles). In some instances, the remote location may be at a distance of even greater than 1500 km (923 miles).

It is contemplated that suitable transportation means includes the use of any of the various means or methods for transporting a fluid from one physical location to another physical location, including, for example, the use of tank trucks, barges or shipping vessels, railroad tanks or tank cars that are equipped with vessels that are suitable for the containment and transportation of fluids, pipelines and any combination of the aforementioned. In the case of the transportation of the polyalkylaromatic compound by pipeline, the polyalkylaromatic compound will be typically delivered to a tank or a vessel for the storage of the polyalkylaromatic compound prior to its use as the transalkylation feed. The alkylation product that is transported to the transalkylation system and used as a transalkylation feed comprises a polyalkylaromatic compound. The term polyalkylaromatic compound may include the compounds of diethylbenzene, triethylbenzene, tetraethylbenzene, pentaethylbenzene, hexaethylbenzene, dipropylbenzene, diisopropylbenzene, tripropylbenzene, triisopropylbenzene, tetrapropylbenzene, tetraisopropylbenzene, pentapropylbenzene, pentaisopropylbenzene, hexapropylbenzene, hexaisopropylbenzene, dibutylbenzene, tributylbenzene, tetrabutylbenzene, pentabutylbenzene, and hexabutylbenzene. The more common of these polyalkylaromatic compounds, however, are diethylbenzene, triethylbenzene, diisopropylbenzene, and triisopropylbenzene.

The amount of polyalkylaromatic compound in the alkylation product that is used as the transalkylation feed may be in the range of from about 5 weight percent to about 80 weight percent, or even to about 90 weight percent, or 95 weight percent or 100 weight percent. It is preferred for the polyalkylaromatic compound content of the alkylation product to be as high as possible, thus, it may be in the range of from 10 weight percent to 80 weight percent, preferably from 15 weight percent to 80 weight percent, and, more preferably from 20 weight percent to 75 weight percent. Monoalkylaromatic compounds may also be present, but the presence of such compounds is not desired and, generally, such compounds are present in an amount less than 75 weight percent of the alkylation product, preferably, less than 50 weight percent, and, most preferably, less than 30 weight percent.

The transalkylation step includes passing the polyalkylaromatic compound with an alkylatable aromatic hydrocarbon, as described hereinbefore, as a transalkylation feed to the transalkylation reactor of a transalkylation system that provides for the transalkylation of the polyalkylaromatic compounds of the alkylation product to yield monoalkylaromatic compounds.

As noted above, the alkylation product preferably includes a substantial amount of polyalkylaromatic compound. More preferably, there is a stoichiometric excess of the polyalkylaromatic compound in the transalkylation system feed. It is also preferred to minimize the amount of olefin compounds that are introduced along with the polyalkylaromatic compound and alkylatable aromatic hydrocarbon to the transalkylation reactor in order to provide favorable transalkylation reaction conditions. Thus, the transalkylation reactor feed should contain an immaterial amount of olefin compounds. It is preferred for the amount of olefins fed to the transalkylation reactor to be less than 20 weight percent of the total weight of the feed to the transalkylation reactor.

As previously noted, the transalkylation system includes a transalkylation reactor for receiving both the polyalkylaromatic compound and an alkylatable aromatic hydrocarbon as a transalkylation feed and yielding a transalkylation product that comprises a major proportion thereof of a monoalkylaromatic compound. The transalkylation reactor may be any suitable apparatus that provides for the contacting of the transalkylation system feed with a transalkylation catalyst under reaction conditions that are suitable for the transalkylation of the polyalkylaromatic compounds with alkylatable aromatic hydrocarbons of the transalkylation system feed. Thus, generally, the transalkylation reactor provides means for contacting the transalkylation feed under suitable transalkylation reaction conditions and may include a vessel that defines a reaction zone containing the transalkylation catalyst.

It is understood herein that the references to the transalkylation system, and, indeed, to the alkylation system and the alkylaromatic reaction system, may or may not include, one or more separation sections as well as one or more reaction sections and all associated or appurtenant equipment. Thus, when referring herein to the transalkylation product it is understood that it may be a transalkylation reactor effluent that has been subjected to a separation or series of separations or a purification or any other form of processing so as to provide a product that is suitable for use as a feed to the alkylaromatic reaction system. Likewise, the references herein to such terminology as alkylation product or similar terminology should be understood to refer to an alkylation reactor effluent itself or to an alkylation reactor effluent which may have been subject to a separation or series of separations or purification or any other form of processing so as to provide a product that is suitable for use as a transalkylation feed to the transalkylation reactor of the transalkylation system.

Any suitable transalkylation catalyst known in the art may be used in the transalkylation step, and it is recognized that catalysts that may suitably be used in the alkylation as described above may also be suitably used as a transalkylation catalyst. Thus, those alkylation catalysts described hereinabove are also the same catalysts that may be used as the transalkylation catalysts. Preferred zeolite transalkylation catalysts are described in detail in U.S. Pat. No. 6,670,517.

The transalkylation reaction conditions are similar to the earlier defined alkylation reaction conditions with the transalkylation reaction temperature being in the range of from 25° C. (77° F.) to 480° C. (896° F.), the transalkylation reaction pressure being in the range of from about atmospheric to about 20,000 kPa (2900 psig), and a weight hourly space velocity (WHSV) based on the sum of the polyalkylaromatic compound and an alkylatable aromatic hydrocarbon feed being in the range of from 0.5 hr⁻¹ to 10 hr⁻¹.

As hereinbefore described, the alkylaromatic compound may undergo further processing or further reactions in an alkylaromatic reaction system to yield one or more products. It is an aspect of the inventive method that the site of the alkylaromatic reaction system may be at a location that is distant and apart from the site of the alkylation system. It is a preferred feature for the transalkylation system and the alkylaromatic reaction system to be a part of an integrated system in which they are linked or joined together at the same geographical location so that the alkylaromatic reaction system may easily receive as its feed the transalkylation product without the need to transport the transalkylation product a great distance. It is preferred for the transalkylation system and the alkylaromatic reaction system to be operatively connected so as to provide for the fluid flow communication between the two systems, and, in particular, for a fluid flow communication that allows for the introduction of the transalkylation product into the alkylaromatic reaction system directly from the transalkylation system.

Specific embodiments of alkylaromatic reaction systems include systems that further react alkylaromatic compounds to produce alkenylaromatic compounds. Examples of these are dehydrogenation systems that produce styrene and hydrogen, SM/PO type processes that produce styrene, and systems that produce alpha-methyl styrene or p-methyl styrene. Other embodiments include systems that further react alkylaromatic compounds to produce phenolic compounds. Examples of these are systems that form cumene hydroperoxide and then decompose the hydroperoxide(s) with an acid catalyst to produce phenol and acetone, and systems that form sec-butylbenzene hydroperoxide and then treat the hydroperoxide(s) with an acid to produce phenol and methyl ethyl ketone.

In addition, it is possible that the alkylaromatic compound will not undergo further processing Some embodiments of alkylaromatic reaction systems are described in further detail hereinafter.

In one embodiment, an alkylaromatic reaction system comprises a dehydrogenation system that yields an alkenylaromatic compound and hydrogen. In this case, the preferred olefin is ethylene; the preferred polyalkylaromatic compound comprises diethylbenzene and/or triethylbenzene; and the preferred transalkylation product is ethylbenzene. In a preferred dehydrogenation system, the dehydrogenation reactor of the dehydrogenation system may be any suitable apparatus that provides for the contacting of the transalkylation product with a dehydrogenation catalyst and, generally, includes a dehydrogenation reactor vessel, which defines a dehydrogenation reaction zone and may contain dehydrogenation catalyst particles that are usually packed together to form a dehydrogenation catalyst bed.

The dehydrogenation catalyst may be any known iron or iron oxide based catalyst that may suitably be used in the dehydrogenation of alkylaromatic compounds. Such dehydrogenation catalysts include those catalysts that comprise iron oxide. The iron oxide of the dehydrogenation catalyst may be in any form and obtained from any source or by any method that provides a suitable iron oxide material for use in the iron oxide based dehydrogenation catalyst. One particularly desirable iron oxide based dehydrogenation catalyst includes potassium oxide and iron oxide.

The iron oxide of the iron oxide based dehydrogenation catalyst may be in a variety of forms including any one or more of the iron oxides, such as, for example, yellow iron oxide (goethite, FeOOH), black iron oxide (magnetite, Fe₃O₄), and red iron oxide (hematite, Fe₂O₃), including synthetic hematite or regenerated iron oxide, or it may be combined with potassium oxide to form potassium ferrite (K₂Fe₂O₄), or it may be combined with potassium oxide to form one or more of the phases containing both iron and potassium as represented by the formula (K₂O)ₓ.(Fe₂O₃)_{y}.

Typical iron oxide based dehydrogenation catalysts comprise from 10 to 98 weight percent iron oxide, calculated as Fe₂O₃, and up to 40 weight percent potassium, calculated as K₂O. The iron oxide based dehydrogenation catalyst may further comprise one or more promoter metals that are usually in the form of an oxide. These promoter metals may be selected from the group consisting of Sc, Y, La, Mo, W, Ce, Rb, Ca, Mg, V, Cr, Co, Ni, Mn, Cu, Zn, Cd, Al, Sn, Bi, rare earths and mixtures of any two or more thereof. Among the promoter metals, preferred are those selected from the group consisting of Ca, Mg, Mo, W, Ce, La, Cu, Cr, V and mixtures of two or more thereof. Most preferred are Ca, Mg, W, Mo, and Ce.

Descriptions of typical iron oxide based dehydrogenation catalysts that may suitably be used as the dehydrogenation catalysts may be found in patent publications that include U.S. Patent Publication No. 2003/0144566 A1; U.S. Pat. No. 5,689,023; U.S. Pat. No. 5,376,613; U.S. Pat. No. 4,804,799; U.S. Pat. No. 4,758,543; U.S. Pat. No. 6,551,958 B1; and EP 0,794,004 B1, all of such patent publications are incorporated herein by reference.

The iron oxide based catalyst is prepared by any method known to those skilled in the art. The iron oxide based dehydrogenation catalyst comprising potassium oxide and iron oxide may, in general, be prepared by combining the components of an iron-containing compound and a potassium-containing compound, shaping these components to form particles, and calcining the particles. The promoter metal-containing compounds may also be combined with the iron-containing and potassium-containing components.

The catalyst components may be formed into particles such as extrudates, pellets, tablets, spheres, pills, saddles, trilobes, tetralobes and the like. One preferred method of making the iron based dehydrogenation catalyst is mixing together the catalyst components with water or a plasticizer, or both, and forming an extrudable paste from which extrudates are formed. The extrudates are then dried and calcined. The calcination is preferably done in an oxidizing atmosphere, such as air, and at temperatures upwardly to 1200° C., but preferably from 500° C. to 1100° C., and, most preferably, from 700° C. to 1050° C.

It is recognized that the dehydrogenation reactor or dehydrogenation reactor system may include more than one dehydrogenation reactor or reaction zone. If more than a single dehydrogenation reactor is used, they may be operated in series or in parallel, or they may be operated independently from each other or under the same or different process conditions.

The dehydrogenation conditions may include a dehydrogenation reactor inlet temperature in the range of from about 500° C. to about 1000° C., preferably, from 525° C. to 750° C., and, most preferably, from 550° C. to 700° C. Thus, the first temperature of the dehydrogenation catalyst bed may range from about 500° C. to about 1000° C., more specifically, from 525° C. to 750° C., and, most specifically, from 550° C. to 700° C.

The reaction pressure is relatively low and may range from vacuum pressure to about 172 kPa (25 psia). The liquid hourly space velocity (LHSV) may be in the range of from about 0.01 hr⁻¹ to about 10 hr⁻¹, and preferably, from 0.1 hr⁻¹ to 2 hr⁻¹. As used herein, the term “liquid hourly space velocity” is defined as the liquid volumetric flow rate of the dehydrogenation feed, for example, ethylbenzene, measured at normal conditions (i.e., 0° C. and 1 kPa absolute), divided by the volume of the catalyst bed, or the total volume of catalyst beds if there are two or more catalyst beds. When styrene is being manufactured by the dehydrogenation of ethylbenzene, it is generally desirable to use steam as a diluent usually in a molar ratio of steam to ethylbenzene in the range of 0.1 to 20. Steam may also be used as a diluent with other dehydrogenatable hydrocarbons.

In another embodiment of an alkylaromatic reaction system, a dehydrogenation system that provides for dehydrogenating a transalkylation product to yield a styrene product may include a so-called SM/PO (styrene monomer/propylene oxide) process system. In this case, the preferred olefin is ethylene; the preferred polyalkylaromatic compound comprises diethylbenzene and/or triethylbenzene; and the preferred transalkylation product is ethylbenzene. An SM/PO process provides for the oxidation of ethylbenzene, for instance, the ethylbenzene of a transalkylation product, with oxygen to yield ethylbenzene hydroperoxide that is reacted with propylene to form propylene oxide and an alcohol by-product that is processed to produce a styrene product.

The SM/PO process is known in the art. In the oxidation step of the SM/PO process, a liquid phase oxidation of ethylbenzene into ethylbenzene hydroperoxide occurs at a temperature in the range of from 100 to 160° C., suitably from 130 to 150° C., and at a pressure in the range of from 1 to 4 bar, suitably from 2 to 3 bar. The oxidation is typically carried out with air as the source of oxygen used for the oxidizing gas, but any fluid that suitably contains oxygen may also be applied. The main by-product formed at this stage of the SM/PO process is acetophenone, which may be hydrogenated into 1-phenylethanol, used in a dehydrating step to produce styrene.

In the epoxidation step of the SM/PO process, ethylbenzene hydroperoxide is reacted with propylene to yield propylene oxide and 1-phenyl ethanol or substituted 1-phenyl ethanol. In such epoxidation step, either a homogeneous catalyst or a heterogeneous catalyst may be applied. As homogeneous catalysts, molybdenum compounds are frequently applied, while catalysts based on titanium on a silica carrier are often used as heterogeneous catalysts.

Conditions under which epoxidation is carried out are known in the art and typically include temperatures in the range of from 75 to 150° C. and pressures up to 80 bar with the reaction medium being in the liquid phase. In the epoxidation step, the alkylaryl hydroperoxide is contacted with olefin in the presence of a catalyst to obtain an oxirane compound and hydroxyalkylaryl. A catalyst that may suitably be used in this step comprises titanium on silica and/or silicate. A preferred catalyst is described in EP-B-345856, the disclosure of which is herein incorporated by reference. The reaction generally proceeds at moderate temperatures and pressures, in particular at temperatures in the range of from 0 to 200° C., preferably in the range from 25 to 200° C. The precise pressure is not critical as long as it suffices to maintain the reaction mixture in a liquid condition. Atmospheric pressure may be satisfactory In general, pressures may be in the range of from about 1 to about 100×10⁵ N/m².

The effluent from the epoxidation step is normally first subjected to a separation treatment to remove the propylene oxide formed, after which the residual stream, containing 1-phenyl ethanol, is suitably subjected to one or more further separation treatments, inter alia to remove ethylbenzene for reuse in an earlier stage of the process. The eventually obtained 1-phenyl ethanol-containing stream is then subjected to the dehydration step.

The dehydration of 1-phenyl ethanol into styrene is also known in the art. It may be carried out in the gas phase or in the liquid phase, or both. Suitable dehydration catalysts include, for instance, acidic materials like alumina, alkali alumina, aluminum silicates and H-type synthetic zeolites. Dehydration conditions are also known and usually include reaction temperatures in the range of from 100 to 210° C. for liquid phase dehydration and in the range of from 210 to 320° C., typically of from 280 to 310° C., for gas phase dehydration. Pressures usually range from 0.1 to bar. Yielded from the dehydration step of the SM/PO process system is the dehydrogenation product, including styrene.

In another embodiment of an alkylaromatic reaction system, a phenol/acetone manufacturing system provides for the conversion of a propylbenzene of the transalkylation product to yield a phenol/acetone product that may comprise phenol or acetone, or both. In this case, the preferred olefin is propylene; the preferred polyalkylaromatic compound comprises diisopropylbenzene and/or triisopropylbenzene; and the preferred transalkylation product is cumene. The phenol/acetone manufacturing system may be any processing system known to those skilled in the art that suitably provides for the conversion of cumene to phenol. Preferred, however, are systems that provide for the oxidation of cumene in air to form cumene hydroperoxide that is cleaved under acidic conditions to form phenol and acetone. Thus, the phenol/acetone manufacturing system may include two steps with one being an oxidation step and the other being a hydroperoxide cleavage step.

The cumene oxidation step is generally conducted in the liquid phase in the presence of an alkaline medium and under oxidation reaction conditions. An oxygen-containing gas, such as air, is passed or bubbled through the cumene at suitable oxidation temperature and pressure conditions. Generally, the oxidation temperature is in the range of from 40° C. to 180° C., and the oxidation pressure is in the range of from 1 to 10,000 kPa (1 to 100 bar) absolute. Any other method that is known to those skilled in the art and is suitable for use in the oxidation of the cumene to cumene hydroperoxide may be used, and one description of such a suitable oxidation method is found in U.S. Pat. No. 6,043,399, which patent is incorporated herein by reference.

The cumene hydroperoxide that is yielded from the first step of the phenol/acetone manufacturing system is then subjected to a hydroperoxide cleavage step whereby the cumene hydroperoxide is converted by the acid catalyzed cleavage reaction in the presence of a suitable acid compound. Typically, sulfuric acid is a suitable acid for catalyzing the cleavage reaction, but other types of acids, and even solid heterogeneous catalysts having an acidic activity, may be used. Generally, the cleavage reaction temperature is in the range of from 40° C. to 180° C., and the cleavage reaction pressure is in the range of from 1 to 10,000 kPa (100 bar) absolute.

Any method that is known to those skilled in the art and that is suitable for the cleavage of cumene hydroperoxide may be used for the second step of the phenol/acetone manufacturing system. Examples of various methods that may suitably be used include those described in U.S. Pat. No. 4,490,565; U.S. Pat. No. 4,567,304; U.S. Pat. No. 5,371,305 and U.S. Pat. No. 6,225,513, all of which are incorporated herein by reference.

A phenol/acetone product is yielded from the phenol/acetone manufacturing system. As it is used herein, the term phenol/acetone product is meant to refer to any one or more of the product streams resulting from the cumene hydroperoxide acid catalyzed cleavage step of the phenol/acetone manufacturing system and may include the entire cleavage reaction effluent stream, which comprises both phenol and acetone, or individual product streams resulting from the separation of the various components of the cleavage reaction effluent stream to provide a phenol product stream, or an acetone product stream, or other streams, such as a residue stream.

In another embodiment, some or all of the cumene may be replaced by sec-butyl benzene. The resulting aromatic product may comprise phenol, and one or more of the product streams may additionally comprise methyl ethyl ketone and acetone. In this case, the preferred olefin is 1-butene and/or 2-butene; the preferred polyalkylaromatic compound comprises polybutylbenzene; and the preferred transalkylation product is sec-butylbenzene.

The inventive method may comprise using an alkylation system for reacting a first alkylatable aromatic hydrocarbon and an olefin under suitable alkylation reaction conditions to thereby form an alkylation product comprising a significant amount of a polyalkylaromatic compound. A transalkylation system is used to react a second alkylatable aromatic hydrocarbon and the polyalkylaromatic compound under suitable transalkylation reaction conditions to thereby form a transalkylation product comprising an alkylaromatic compound. A dehydrogenation system may be used in dehydrogenating the transalkylation product to thereby yield a dehydrogenation product. The dehydrogenation system of the inventive method may further be located at a remote location from the alkylation system, and the transalkylation system may be coupled with the dehydrogenation system, and, thus, the transalkylation system is separated from the alkylation system. The first and second alkylatable aromatic hydrocarbons may comprise benzene

The inventive method may comprise passing an alkylation feed comprising benzene and ethylene to an alkylation system by which a polyethylbenzene product is yielded that comprises polyethylbenzene which step is followed by passing benzene and the polyethylbenzene product as a transalkylation feed to a transalkylation system by which a transalkylation product that comprises ethylbenzene is yielded. The transalkylation product is then passed as a dehydrogenation feed to a dehydrogenation system by which a styrene product is yielded that comprises styrene. This embodiment of the invention may further include the transporting of the polyethylbenzene product by transportation means to the transalkylation system that is located at a remote location from the alkylation system where the transalkylation step is performed. The transalkylation system may also be coupled with the dehydrogenation system, and, thus, the transalkylation system may be separated from the alkylation system.

The inventive method may comprise a method of making a vinyl aromatic compound from a first alkylatable aromatic hydrocarbon and an olefin by providing an alkylaromatic feed for use in an alkylaromatic dehydrogenation unit that is located at a remote location from a polyalkylation unit, wherein the alkylaromatic dehydrogenation unit is coupled with a transalkylation unit. This method comprises alkylating said first alkylatable aromatic hydrocarbon under polyalkylation reaction conditions to thereby form a polyalkylaromatic product, comprising a significant amount of polyalkylaromatic compounds. The polyalkylaromatic product is transported to the remote location where it is utilized as a transalkylation feed to the transalkylation unit, which thereby yields the alkylaromatic feed, and the alkylaromatic feed is utilized as a dehydrogenation feed to the alkylaromatic dehydrogenation unit, which thereby yields a dehydrogenation product.

Reasonable variations, modifications and adaptations may be made within the scope of the described disclosure and the appended claims without departing from the scope of the invention.

That which is claimed is:

1. A method, comprising:
contacting an alkylatable aromatic hydrocarbon and a polyalkylaromatic compound in a transalkylation system to yield an alkylaromatic compound,
wherein at least a portion of the polyalkylaromatic compound has been formed by contacting an alkylatable aromatic hydrocarbon and an olefin at a location that is remote from the transalkylation system.

2. A method as claimed in claim 1 further comprising further reacting the alkylaromatic compound to yield an alkenylaromatic compound.

3. A method as claimed in claim 2 wherein the further reaction comprises a dehydrogenation reaction yielding the alkenylaromatic compound and hydrogen.

4. A method as claimed in claim 1 further comprising further reacting the alkylaromatic compound to yield a phenolic compound.

5. A method as claimed in claim 1 wherein the alkylatable aromatic hydrocarbon comprises benzene.

6. A method as claimed in claim 1 wherein the olefin comprises ethylene.

7. A method as claimed in claim 1 wherein the olefin comprises propylene.

8. A method as claimed in claims 2 or 4 wherein the transalkylation system is in fluid communication with a reaction system in which the further reaction is carried out.

9. A method, comprising:
reacting an alkylaromatic compound to yield an alkenylaromatic compound,
wherein at least a portion of the alkylaromatic compound has been formed in a transalkylation system by contacting an alkylatable aromatic hydrocarbon and a polyalkylaromatic compound that has been formed at a location that is remote from the transalkylation system.

10. A method as claimed in claim 9 wherein the reaction comprises a dehydrogenation reaction that yields the alkenylaromatic compound and hydrogen.

11. A method, comprising:
reacting an alkylaromatic compound to yield a phenolic compound,
wherein at least a portion of the alkylaromatic compound has been formed in a transalkylation system by contacting an alkylatable aromatic hydrocarbon and a polyalkylaromatic compound that has been formed at a location that is remote from the transalkylation system.
